(19) United States
(12) Patent Application Publication
(10) Pub. No.: US 2008/0301836 A1
(43) Pub. Date: Dec. 4, 2008

(54) Selection of transcription factor variants

(75) Inventors: Karen S. Century, Albany, CA (US); T. Lynne Reuber, San Mateo, CA (US); Katrin Jakob, Alameda, CA (US); Oliver J. Ratcliffe, Oakland, CA (US)

Correspondence Address: Mendel Biotechnology, Inc., 3935 Point Eden Way, Hayward, CA, 94545, US

(73) Assignee: Mendel Biotechnology, Inc., Hayward, CA (US)

(21) Appl. No.: 12/154,154

(22) Filed: May 19, 2008

Related U.S. Application Data
(60) Provisional application No. 60/930,870, filed on May 17, 2007.

(51) Int. Cl. C12N 15/11; A01H 5/00

(52) U.S. Cl. 800/279; 800/278; 800/298

(57) ABSTRACT

The invention relates to a method for selection of modified plant transcription factor polypeptides, polynucleotides that encode them, and methods of producing transgenic plants having advantageous properties, including increased biotic resistance and abiotic stress tolerance, as compared to wild-type or control plants. Without modifications, the transcription factor sequences, when overexpressed in plants, often produce adverse morphological and developmental effects. The disclosed method allows selection of modifications that mitigate these adverse morphological and developmental effects.

This application claims the benefit of U.S. Provisional Patent Application 60/930,870, filed May 17, 2007 (pending), the entire contents of which are hereby incorporated by reference.

### JOINT RESEARCH AGREEMENT

The claimed invention, in the field of functional genomics and the characterization of plant genes for the improvement of plants, was made by or on behalf of Mendel Biotechnology, Inc. and Monsanto Company as a result of activities undertaken within the scope of a joint research agreement in effect on or before the date the claimed invention was made.

### FIELD OF THE INVENTION

The present invention relates to plant genomics and plant improvement.

### BACKGROUND OF THE INVENTION

Enhanced expression of regulatory proteins such as transcription factors can produce a number of beneficial effects in transgenic plants, including disease resistance, abiotic stress tolerance, improved water use efficiency, improved nutrient use efficiency, faster seed germination, and altered chemical composition. However, overexpression of transcription factors can also cause negative phenotypes, such as reduced plant growth, undesirable alterations in flowering time, and reduced seed yield. One method for reducing such negative side effects is to restrict the spatial or temporal expression of the transcription factor, using a tissue-specific or inducible promoter. However, this strategy is not completely effective in all cases. A second strategy is to engineer the transcription factor protein to alter the range of target genes which it regulates. Alterations in transcription factor proteins can alter either DNA binding specificity or interactions with particular co-factors, and changes in either of these properties can alter the target specificity and therefore the phenotypic effects of transcription factor expression. The present invention provides a method for selecting transcription factor variants that produce desirable stress tolerance phenotypes with reduced negative effects of overexpression. The method is demonstrated with the AP2 domain transcription factors TDR4 and Pti4. These transcription factors produce disease resistance when overexpressed, but produce negative morphological effects such as stunting, delayed flowering, and infertility when constitutively expressed. However, the method can be generalized to other transcription factors from other gene families.

### SUMMARY OF THE INVENTION

The invention pertains to a method for producing a plant that has greater biotic stress resistance and/or greater abiotic stress tolerance than a control plant, such as a wild-type plant or a non-transformed plant of the same species. The former plant with greater biotic stress resistance or abiotic stress tolerance comprises a mutant form of a transcription factor sequence, and the former plant also has fewer or reduced adverse morphological or developmental effects than a second control plant that constitutively overexpresses the transcription factor sequence. This method is practiced by generating a two-component expression system that comprises two nucleic acid constructs. The first nucleic acid construct, the target construct, encodes a transcription factor polypeptide. The target construct may comprise a LexA operator in front of the transcription factor gene. The second nucleic acid construct, or activator construct, encodes a steroid-binding domain of the glucocorticoid receptor.

Transgenic plant lines are generated by introducing the two constructs into plants, and transgenic plants are selected that comprise both the target nucleic acid construct and the activator nucleic acid construct are homozygous for both constructs.

In one embodiment, the target plant is generated by introducing the activator nucleic acid construct into a first plant, a second plant is selected that is homozygous for the activator nucleic acid construct, the second plant is then transformed with the target nucleic acid construct to generate a third plant, and a fourth plant is selected that is homozygous for both the activator and target nucleic acid constructs.

The transgenic plant lines are mutagenized to produce a pool of mutagenized transgenic plant lines comprising sequence variants of the transcription factor polypeptide. One or more of the mutagenized transgenic plant lines are then selected that have both greater biotic or abiotic stress tolerance than the first control plant that does not overexpress the transcription factor polypeptide. The mutagenized transgenic plant lines also have fewer or reduced adverse morphological or developmental effects as compared to the second control plant that constitutively overexpresses the transcription factor polypeptide.

### BRIEF DESCRIPTION OF THE SEQUENCE LISTING AND FIGURES

The Sequence Listing provides exemplary polynucleotide and polypeptide sequences of the invention. The traits associated with the use of the sequences are included in the Examples.

CD-ROMs Copy 1 and Copy 2, and the CRF copy of the Sequence Listing under CFR Section 1.821(e), are read-only memory computer-readable compact discs. Each contains a copy of the Sequence Listing in ASCII text format. The Sequence Listing is named “MBI0081US_ST25.txt”, the electronic file of the Sequence Listing contained on each of these CD-ROMs was created on May 16, 2008, and is 1,924 kilobytes in size. The copies of the Sequence Listing on the CD-ROM discs are hereby incorporated by reference in their entirety.

FIG. 1 shows a conservative estimate of phylogenetic relationships among the orders of flowering plants (modified from Soltis et al. (1997) Ann. Missouri Bot. Gard. 84: 1-49). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales.

FIG. 1 was adapted from Daly et al. (2001) Plant Physiol. 127: 1328-1333.

FIG. 2 shows a phylogenic dendogram depicting phylogenetic relationships of higher plant taxa, including clades containing tomato and Arabidopsis; adapted from Ku et al. (2000) Proc. Natl. Acad. Sci. USA 97: 9121-9126; and Chase et al. (1993) Ann. Missouri Bot. Gard. 80: 528-580.

FIG. 3 shows a two-component system for dexamethasone-inducible expression of a transcription factor. On the left is an activator construct, consisting of a constitutively-expressed chimeric transcriptional activator comprising the LexA DNA binding domain fused to the GAL4 activation domain and the steroid binding domain of the glucocorticoid receptor. This artificial activator is constitutively expressed, but can only enter the nucleus and activate transcription in the presence of dexamethasone. In addition, to test for promoter and activator functioning the activator construct also contains a LexA operator (from plasmid p8op-lacZ; Clontech Laboratories, Inc., Mountain View, Calif.) plus TATA sequence fused to the Green Fluorescent Protein (GFP) gene. On the right is the target construct, consisting of a LexA operator in front of the transcription factor gene (TF gene) of interest. The target construct is transformed into a plant strain carrying the activator construct.

### DETAILED DESCRIPTION

The present invention relates to polynucleotides and polypeptides for modifying phenotypes of plants, particularly those associated with increased abiotic stress tolerance and increased yield with respect to a control plant (for example, a wild-type plant). Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### Definitions

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al. (1976) Glossary of Genetics and Cytogenetics: Classical and Molecular, 4th ed., Springer Verlag, Berlin). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

A transcription factor that may be used mutagenized used to produce transformed plants with increased resistance to biotic stress or increased tolerance to biotic stress will have a minimum percentage identity to the listed polypeptide sequences. Functional transcription factors of the invention may exhibit a degree of sequence homology such as at least about 56% sequence identity, or at least about 58% sequence identity, or at least about 60% sequence identity, or at least about 65%, or at least about 67%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% amino acid residue sequence identity, to a polypeptide provided in the Sequence Listing, e.g., SEQ ID NOs: 2, 4, or sequences that are orthologous to SEQ ID NOs: 2 or 4, or SEQ ID NO: 12, 14, 16, 18, 20, 22, 24, 26, 28, 30, 32, 34, 36, 38 or 40, or 41-72, or 74, 76, 78, 80, 82, 84, 86, 88, 90, 92, 94, or 96, or any of SEQ ID NO: 2n−1, where n=56-487.

“Alignment” refers to a number of nucleotide bases or amino acid residue sequences aligned by lengthwise comparison so that components in common (i.e., nucleotide bases or amino acid residues at corresponding positions) may be visually and readily identified. The fraction or percentage of components in common is related to the homology or identity between the sequences. An alignment may suitably be determined by means of computer programs known in the art, such as MACVECTOR software (1999) (Acceirys, Inc., San Diego, Calif.).

A “conserved domain” or “conserved region” as used herein refers to a region in heterologous polynucleotide or polypeptide sequences where there is a relatively high degree of sequence identity between the distinct sequences. An AP2 domain, or “B-box zinc finger” domain”, such as is found in a polypeptide member of AP2 and B-box zinc finger families, respectively, are examples of conserved domains. With respect to polynucleotides encoding presently disclosed polypeptides, a conserved domain is preferably at least nine base pairs (bp) in length. A conserved domain with respect to presently disclosed polypeptides refers to a domain within a polypeptide family that exhibits a higher degree of sequence homology, such as at least about 56% sequence identity, or at least about 58% sequence identity, or at least about 60% sequence identity, or at least about 65%, or at least about 67%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% amino acid residue sequence identity, to a conserved domain of a polypeptide of the invention (e.g., SEQ ID NOs: 2, 4, or sequences that are orthologous to SEQ ID NOs: 2 or 4, or any of SEQ ID NO: 2n−1, where n=56-487). Sequences that possess or encode for conserved domains that meet these criteria of percentage identity, and that have comparable biological activity to the present polypeptide sequences, for example, as members of the same clade polypeptides, such as sequences closely related to TDR4, SEQ ID NO: 2 or Pti4, SEQ ID NO: 4 are encompassed by the invention. A fragment or domain can be referred to as outside a conserved domain, outside a consensus sequence, or outside a consensus DNA-binding site that is known to exist or that exists for a particular polypeptide class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (see, for example, Riechmann et al. (Riechmann et al. (2000a) Science 290, 2105-2110, and Riechmann and Ratcliffe (2000b) Curr. Opin. Plant Biol. 3, 423-434). Thus, by using alignment methods well known in the art, the conserved domains of the plant polypeptides, for example, for the AP2 family of transcription factors, or the B-box zinc finger proteins (Putterill et al. (1995) Cell 80: 847-857), may be determined.

The conserved domains for many of the polypeptide sequences of the invention are listed in Tables 1 and 2. Also, the polypeptides of Tables 1 and 2 have conserved domains specifically indicated by amino acid coordinate start and stop sites. A comparison of the regions of these polypeptides allows one of skill in the art (see, for example, Reeves and Nissen (1990) J. Biol. Chem. 265, 8573-8582) to identify domains or conserved domains for any of the polypeptides listed or referred to in this disclosure.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985) Nature 313: 402-404, Sambrook et al. (1989) Molecular Cloning: A Laboratory Manual, 2nd Ed., Cold Spring Harbor Laboratory, Cold Spring Harbor, N.Y., and by Haymes et al. (1985) Nucleic Acid Hybridization: A Practical Approach, IRL Press, Washington, D.C., which references are incorporated herein by reference.

In general, stringency is determined by the temperature, ionic strength, and concentration of denaturing agents (e.g., formamide) used in a hybridization and washing procedure (for a more detailed description of establishing and determining stringency, see the section “Identifying Polynucleotides or Nucleic Acids by Hybridization”, below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known related polynucleotide sequences. Numerous variations are possible in the conditions and means by which nucleic acid hybridization can be performed to isolate related polynucleotide sequences having similarity to sequences known in the art and are not limited to those explicitly disclosed herein. Such an approach may be used to isolate polynucleotide sequences having various degrees of similarity with disclosed polynucleotide sequences, such as, for example, encoded transcription factors having 56% or greater identity with the conserved domain of disclosed sequences.

The invention also pertains to a nucleic acid construct, or a transformed plant comprising such a construct, where the construct comprises a nucleic acid sequence found in the Sequence Listing, or a sequence that is homologous to any of these sequences and that functions in a similar manner, or a sequence that hybridizes to any of these sequences under stringent conditions. Stingent conditions may comprise at least 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. It is known in the art that hybridization techniques using a known nucleic acid as a probe under highly stringent conditions will identify structurally similar nucleic acids.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent polypeptides. Thus, it will be readily appreciated by those of skill in the art, that any of a variety of polynucleotide sequences is capable of encoding the polypeptides and homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the polypeptide is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544). Amino acid substitutions outside of the identified functional conserved domains are unlikely to greatly affect regulatory activity of the present transcription factors.

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the polynucleotides listed in the Sequence Listing. Exemplary fragments also include fragments that comprise a region that encodes an conserved domain of a polypeptide. Exemplary fragments also include fragments that comprise a conserved domain of a polypeptide. Exemplary fragments include fragments that comprise an conserved domain of a polypeptide such as a domain associated with a function of the polypeptide (e.g., a domain that binds to a DNA promoter region, an activation domain, or a domain for protein-protein interactions, etc.).

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

The invention also encompasses production of DNA sequences that encode polypeptides and derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding polypeptides or any fragment thereof.

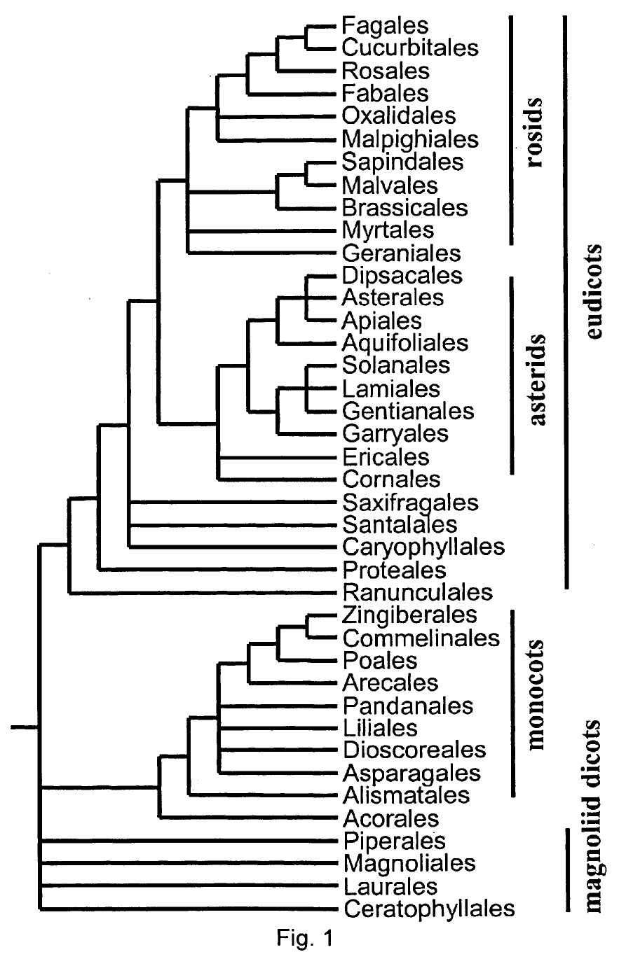

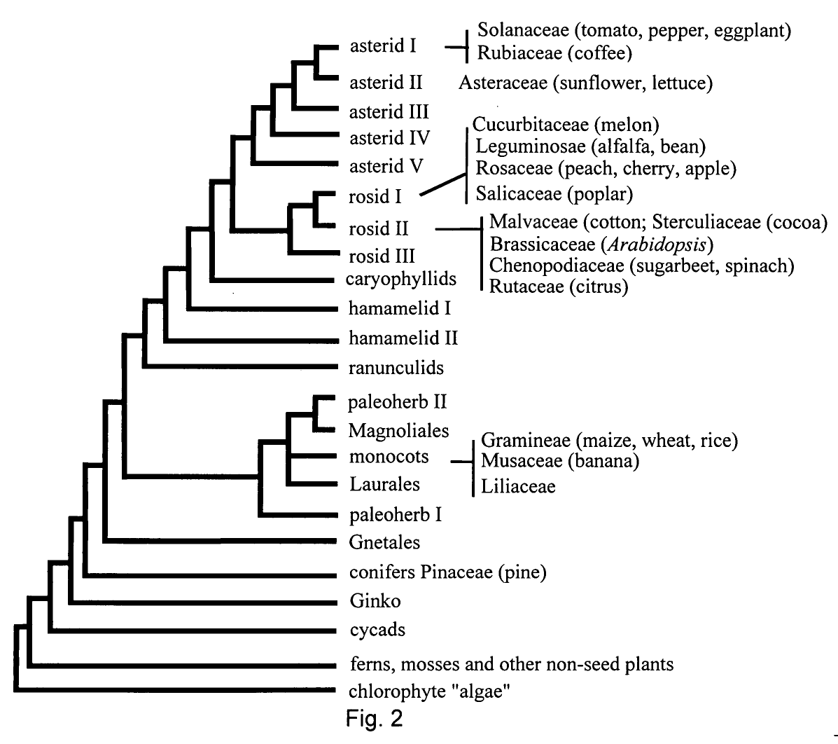

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see for example, FIG. 1, adapted from Daly et al. (2001) supra, FIG. 2, adapted from Ku et al. (2000) supra; and see also Tudge (2000) in The Variety of Life, Oxford University Press, New York, N.Y. pp. 547-606.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the controlled expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a polypeptide's expression is altered, e.g., in that it has been knocked out, overexpressed, or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, color, stem diameter, leaf size, leaf dimension, leaf density, internode distance, branching, root branching, number and form of inflorescences, and other macroscopic characteristics, and the individual plants are not readily distinguishable based on morphological characteristics alone.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the transcript profile of a particular polypeptide in a suspension cell is the expression levels of a set of genes in a cell knocking out or overexpressing that polypeptide compared with the expression levels of that same set of genes in a suspension cell that has normal levels of that polypeptide. The transcript profile can be presented as a list of those genes whose expression level is significantly different between the two treatments, and the difference ratios. Differences and similarities between expression levels may also be evaluated and calculated using statistical and clustering methods.

“Ectopic expression or altered expression” in reference to a polynucleotide indicates that the pattern of expression in, e.g., a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more polypeptides are under the control of a strong promoter (e.g., the cauliflower mosaic virus 35S transcription initiation region). Overexpression may also under the control of an inducible or tissue specific promoter. Thus, overexpression may occur throughout a plant, in specific tissues of the plant, or in the presence or absence of particular environmental signals, depending on the promoter used.

Overexpression may take place in plant cells normally lacking expression of polypeptides functionally equivalent or identical to the present polypeptides. Overexpression may also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors possess an conserved domain. The transcription factors also comprise an amino acid subsequence that forms a transcription activation domain that regulates expression of one or more abiotic stress tolerance genes in a plant when the transcription factor binds to the regulating region.

“Yield” or “plant yield” refers to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency.

“Planting density” refers to the number of plants that can be grown per acre. For crop species, planting or population density varies from a crop to a crop, from one growing region to another, and from year to year. Using corn as an example, the average prevailing density in 2000 was in the range of 20,000-25,000 plants per acre in Missouri, USA. A desirable higher population density (a measure of yield) would be at least 22,000 plants per acre, and a more desirable higher population density would be at least 28,000 plants per acre, more preferably at least 34,000 plants per acre, and most preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al. (2003) U.S. Patent Application No. 20030101479). A desirable higher population density for each of these examples, as well as other valuable species of plants, would be at least 10% higher than the average prevailing density or yield.

Regarding the terms “biotrophs” and “necrotrophs”, plant pathogens fall into these two major classes (reviewed in Oliver and Ipcho (2004) Mol. Plant. Pathol. 5, 347-352). Biotrophic pathogens obtain energy by parasitizing living plant tissue, while necrotrophs obtain energy from dead plant tissue. Examples of biotrophs include the powdery mildews, rusts, and downy mildews; these pathogens can only grow in association with living plant tissue, and parasitize plants through intracellular feeding structures called haustoria. Examples of necrotrophs include Sclerotinia sclerotiorum (white mold), Botrytis cinerea (grey mold), and Cochliobolus heterostrophus (Southern corn leaf blight). The general pathogenic strategy of necrotrophs is to kill plant tissue through toxins and lytic enzymes, and live off the released nutrients. Pathologists also recognize a third class of pathogens, called hemibiotrophs: these pathogens have an initial biotrophic stage, followed by a necrotrophic stage once a parasitic association with plant cells has been established. In general, different defense responses have been found to be induced in plants in response to attack by a biotrophic or necrotrophic pathogen. Infection by biotrophic pathogens often induces defense responses mediated by the plant hormone salicylic acid, while attack by a necrotrophic pathogen often induces defense responses mediated by coordinated action of the hormones ethylene and jasmonate.

### DESCRIPTION OF THE SPECIFIC EMBODIMENTS

### Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding motif (see, for example, Riechmann et al. (2000a) supra). The plant transcription factors of the present invention belong to various transcription factor families, such as the AP2 transcription factor family and include putative transcription factors.

Generally, transcription factors are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to osmotic stresses. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, e.g., mutation reactions, PCR reactions, or the like; as substrates for cloning e.g., including digestion or ligation reactions; and for identifying exogenous or endogenous modulators of the transcription factors. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode polypeptides that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) Genes Development 11: 3194-3205, and Peng et al. (1999) Nature 400: 256-261. In addition, many others have demonstrated that an Arabidopsis transcription factor expressed in an exogenous plant species elicits the same or very similar phenotypic response. See, for example, Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500.

In another example, Mandel et al. (1992b) Cell 71-133-143, and Suzuki et al. (2001) Plant J. 28: 409-418, teach that a transcription factor expressed in another plant species elicits the same or very similar phenotypic response of the endogenous sequence, as often predicted in earlier studies of Arabidopsis transcription factors in Arabidopsis (see Mandel (1992a) Nature 360: 273-277; Suzuki et al. (2001) supra). Other examples include Miller et al. (2001) Plant J. 28: 169-179; Kim et al. (2001) Plant J. 25: 247-259; Kyozuka and Shimamoto (2002) Plant Cell Physiol. 43: 130-135; Boss and Thomas (2002) Nature, 416: 847-850; He et al. (2000) Transgenic Res. 9: 223-227; and Robson et al. (2001) Plant J. 28: 619-631.

In yet another example, Gilmour et al. (1998) Plant J. 16: 433-442, teach an Arabidopsis AP2 transcription factor, CBF1, which, when overexpressed in transgenic plants, increases plant freezing tolerance. Jaglo et al. (2001) Plant Physiol. 127: 910-917, further identified sequences in Brassica napus which encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, PKK/RPAGRxKFxETRHP (SEQ ID NO: 9) and DSAWR (SEQ ID NO: 10), which bracket the AP2/EREBP DNA binding domains of the proteins and distinguish them from other members of the AP2/EREBP protein family. (Jaglo et al. (2001) supra)

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (e.g., by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene and other genes in the MYB family have been shown to control anthocyanin biosynthesis through regulation of the expression of genes known to be involved in the anthocyanin biosynthetic pathway (Bruce et al. (2000) Plant Cell 12: 65-79; and Borevitz et al. (2000) Plant Cell 12: 2383-2393). Further, global transcript profiles have been used successfully as diagnostic tools for specific cellular states (e.g., cancerous vs. non-cancerous; Bhattacharjee et al. (2001) Proc. Natl. Acad. Sci. USA 98: 13790-13795; and Xu et al. (2001) Proc. Natl. Acad. Sci. USA 98: 15089-15094). Consequently, it is evident to one skilled in the art that similarity of transcript profile upon overexpression of different transcription factors would indicate similarity of transcription factor function.

Polypeptides and Polynucleotides of the Invention

The present invention includes putative transcription factors (TFs), and isolated or recombinant polynucleotides encoding the polypeptides, or novel sequence variant polypeptides or polynucleotides encoding novel variants of polypeptides derived from the specific sequences provided in the Sequence Listing; the recombinant polynucleotides of the invention may be incorporated in expression vectors for the purpose of producing transformed plants. Also provided are methods for modifying yield from a plant by modifying the mass, size or number of plant organs or seed of a plant by controlling a number of cellular processes, and for increasing a plant's resistance to abiotic stresses. These methods are based on the ability to alter the expression of critical regulatory molecules that may be conserved between diverse plant species. Related conserved regulatory molecules may be originally discovered in a model system such as Arabidopsis and homologous, functional molecules then discovered in other plant species. The latter may then be used to confer increased yield or abiotic stress tolerance in diverse plant species.

Exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known polypeptides. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known polypeptides.

Additional polynucleotides of the invention were identified by screening Arabidopsis thaliana and/or other plant cDNA libraries with probes corresponding to known polypeptides under low stringency hybridization conditions. Additional sequences, including full length coding sequences, were subsequently recovered by the rapid amplification of cDNA ends (RACE) procedure using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

Many of the sequences in the Sequence Listing, derived from diverse plant species, have been ectopically expressed in overexpressor plants. The changes in the characteristic(s) or trait(s) of the plants were then observed and found to confer increased yield and/or increased abiotic stress tolerance. Therefore, the polynucleotides and polypeptides can be used to improve desirable characteristics of plants.

The polynucleotides of the invention were also ectopically expressed in overexpressor plant cells and the changes in the expression levels of a number of genes, polynucleotides, and/or proteins of the plant cells observed. Therefore, the polynucleotides and polypeptides can be used to change expression levels of genes, polynucleotides, and/or proteins of plants or plant cells.

### The G11792 Clade of Transcription Factors

We first identified G1792 (AT3G23230; SEQ ID NO: 169 and 170 of U.S. Pat. No. 7,193,129) as a transcription factor in the sequence of BAC clone K14B15 (AB025608, gene K14B15.14). We have assigned the name TRANSCRIPTIONAL REGULATOR OF DEFENSE RESPONSE 1 (TDR1) to this gene, based on its apparent role in disease responses. The G1792 transcription factor and closely related proteins in the G1792 clade contain a single AP2 domain and belongs to the ERF class of AP2 proteins. The G11792 clade includes TDR4 and other transcription factors found in Table 1; a number of these sequences have been shown to confer increased disease tolerance in plants when overexpressed (see, for example, patent publications US20050155117A1, and particularly Table 15 of PCT/US2006/34615).

The G1792 clade of transcription factors is characterized by at least two domains responsible for transcription regulatory activity, the AP2 DNA binding domain and the EDLL activation domain (Table 1). Conservative mutations in these domains will result in G1792 clade member polypeptides having activity transcription regulatory activity and functions similar to those performed by G11792 in plant cells. Although all conservative amino acid substitutions in these domains will not necessarily result in the clade member polypeptides having regulatory activity, those of ordinary skill in the art would expect that many of these conservative substitutions would result in a protein having the regulatory activity. Further, amino acid substitutions outside of these two functional domains and other conserved domains in the G1792 clade proteins are unlikely to greatly affect activity the regulatory activity of the G1792 polypeptides.

### The G28 Clade of Transcription Factors, Including Pti4

G28 (SEQ ID NO: 17 and 18 of U.S. Pat. No. 6,664,446) corresponds to AtERF1 (GenBank accession number AB008103) (Fujimoto et al. (2000) Plant Cell 12: 393-404). G28 appears as gene At4g17500 in the annotated sequence of Arabidopsis chromosome 4 (AL161546.2). G28 has been shown to confer resistance to both necrotrophic and biotrophic pathogens. The G28 polypeptide (SEQ ID NO: 18 of U.S. Pat. No. 6,664,446) is a member of the B-3a subgroup of the ERF subfamily of AP2 transcription factors, defined as having a single AP2 domain and having specific residues in the DNA binding domain that distinguish this large subfamily (65 members) from the DREB subfamily. AtERF1 is apparently orthologous to the AP2 transcription factor Pti4 (SEQ ID NO: 4 of the present application), identified in tomato, which has been shown by Martin and colleagues to function in the Pto disease resistance pathway, and to confer broad-spectrum disease resistance when overexpressed in Arabidopsis (Zhou et al. (1997) EMBO J. 16: 3207-3218; Gu et al. (2000) Plant Cell 12: 771-786; Gu et al. (2002) Plant Cell 14: 817-831).

In addition to the AP2 DNA binding domain, the G28 clade of transcription factors is characterized by a potential acidic activation domain and a potential nuclear localization domain. In Pti4, these domains span amino acids 32-56 and 177-199, approximately and respectively. In G28, these domains span amino acids of about 66-90 and 219-238, approximately and respectively. Conservative mutations in these domains will result in G28 clade member polypeptides having activity transcription regulatory activity and functions similar to those performed by G28 or Pti4 in plant cells. Although all conservative amino acid substitutions in these domains will not necessarily result in the clade member polypeptides having regulatory activity, those of ordinary skill in the art would expect that many of these conservative substitutions would result in a protein having the regulatory activity. Further, amino acid substitutions outside of these functional domains and other conserved domains in these proteins are unlikely to greatly affect activity the regulatory activity of the G28 polypeptides.

Tables 1-2 list a number of polypeptides of the invention and include the amino acid residue coordinates for the conserved domains, the conserved domain sequences of the respective polypeptides; the identity in percentage terms to the conserved domain of the lead Arabidopsis sequence (the first transcription factor listed in each table), and whether the given sequence in each row was shown to confer increased biomass and yield or stress tolerance in plants (+) or has thus far not been shown to confer stress tolerance (−) for each given promoter::gene combination in our experiments. Percentage identities to the sequences listed in Tables 1-2 were determined using BLASTP analysis with defaults of wordlength (W) of 3, an expectation (E) of 10, and the BLOSUM62 scoring matrix Henikoff & Henikoff (1992). When the conserved domain sequences found in Tables 1-2 are optimally aligned using the BLOSUM62 matrix, a gap existence penalty of 11, and a gap extension penalty of 1, similar conserved domains may be identified by virtue of having a minimum specified percentage identity. Said minimum percentage identity may be determined by the percentage identities found within a given clade of transcription factors. Examples of percentage identities to Arabidopsis sequences that are clade members are provided in Tables 1-2, although it is anticipated and expected that other percentage identities may be determined by related lade sequences to another Arabidopsis sequence, or a sequence from another plant species, where that sequence is a functional lade member.

TABLE 1

Conserved domains of TDR4 (G1792 clade member, TF family
AP2) and some closely related sequences in the G1792 clade

| | AP2 and | | | | | | | Disease |
|---|---|---|---|---|---|---|---|---|
| GID No./ | EDLL | | SEQ ID | % ID to | | SEQ ID | % ID to | resistance |
| Species | Domains in | | NO: of | AP2 | | NO: of | EDLL | observed in |
| SEQ ID | Amino Acid | | AP2 | Domain | EDLL | EDLL | Domain | overexpressors |
| NO: | Coordinates | AP2 domain | domain | of TDR4 | Domain | domain | of TDR4 | tested to date |
| At/TDR4 | 16-80; 100- | EQGKYRGVRRR | 41 | 100% | VFEFEY | 57 | 100% | B, E, S |
| (G30) | 115 | PWGKYAAEIRD | | | LDDSVL | | | |
| (2) | | SRKHGERVWLG | | | DELL | | | |
| | | TFDTAEDAARA | | | | | | |
| | | YDRAAYSMRG | | | | | | |
| | | KAAILNFPHEY | | | | | | |
| At/G1795 | 11-75; 104- | EHGKYRGVRRR | 42 | 93% | VFEFEY | 58 | 93% | B, E, S |
| (12) | 119 | PWGKYAAEIRD | | | LDDSVL | | | |
| | | SRKHGERVWLG | | | EELL | | | |
| | | TFDTAEEAARA | | | | | | |
| | | YDQAAYSMRG | | | | | | |
| | | QAAILNFPHEY | | | | | | |
| At/G1791 | 10-74; 108- | NEMKYRGVRK | 43 | 85% | VIEFEYL | 59 | 81% | B, E, S |
| (14) | 123 | RPWGKYAAEIR | | | DDSLLE | | | |
| | | DSARHGARVWL | | | ELL | | | |
| | | GTFNTAEDAAR | | | | | | |
| | | AYDRAAFGMR | | | | | | |
| | | GQRAILNFPHEY | | | | | | |
| Os/G3381 | 14-78; 109- | LVAKYRGVRRR | 44 | 84% | PIEFEYL | 60 | 78% | E, S |
| (16) | 124 | PWGKFAAEIRD | | | DDHVL | | | |
| | | SSRHGVRVWLG | | | QEML | | | |
| | | TFDTAEEAARA | | | | | | |
| | | YDRSAYSMRGA | | | | | | |
| | | NAVLNFPADA | | | | | | |
| Os/G3383 | 9-73; 101- | TATKYRGVRRR | 45 | 81% | KIEFEYL | 61 | 85% | |
| (18) | 116 | PWGKFAAEIRD | | | DDKVL | | | |
| | | PERGGARVWLG | | | DDLL | | | |
| | | TFDTAEEAARA | | | | | | |
| | | YDRAAYAQRG | | | | | | |
| | | AAAVLNFPAAA | | | | | | |
| Zm/G3739 | 13-77; 107- | EPTKYRGVRRR | 46 | 79% | VIELEY | 62 | 68% | E |
| (20) | 122 | PWGKYAAEIRD | | | LDDEVL | | | |
| | | SSRHGVRIWLG | | | QEML | | | |
| | | TFDTAEEAARA | | | | | | |
| | | YDRSAYSMRGA | | | | | | |
| | | NAVLNFPEDA | | | | | | |
| Zm/G3517 | 13-77; 103- | EPTKYRGVRRR | 47 | 78% | VIEFEYL | 63 | 75% | E, S |
| (22) | 118 | PWGKYAAEIRD | | | DDEVLQ | | | |
| | | SSRHGVRIWLG | | | EML | | | |
| | | TFDTAEEAARA | | | | | | |
| | | YDRSANSMRGA | | | | | | |
| | | NAVLNFPEDA | | | | | | |
| Os/G3737 | 8-72; 101- | AASKYRGVRRR | 48 | 77% | KVELVY | 64 | 78% | E |
| (24) | 116 | PWGKFAAEIRD | | | LDDKVL | | | |
| | | PERGGSRVWLG | | | DELL | | | |
| | | TFDTAEEAARA | | | | | | |
| | | YDRAAFAMKG | | | | | | |
| | | AMAVLNFPGRT | | | | | | |
| Gm/G3520 | 14-78; 109- | EEPRYRGVRRR | 49 | 76% | VIEFECL | 65 | 62% | E, S |
| (26) | 124 | PWGKFAAEIRD | | | DDKLLE | | | |
| | | PARHGARVWLG | | | DLL | | | |
| | | TFLTAEEAARA | | | | | | |
| | | YDRAAYEMRG | | | | | | |
| | | ALAVLNFPNEY | | | | | | |
| Os/G3380 | 18-82; 103- | ETTKYRGVRRR | 50 | 76% | VIELECL | 66 | 62% | E |
| (28) | 118 | PSGKFAAEIRDS | | | DDQVL | | | |
| | | SRQSVRVWLGT | | | QEML | | | |
| | | FDTAEEAARAY | | | | | | |
| | | DRAAYAMRGH | | | | | | |
| | | LAVLNFPAEA | | | | | | |
| Zm/G3794 | 6-70; 102- | EPTKYRGVRRR | 51 | 75% | VIELECL | 67 | 62% | |
| (30) | 117 | PSGKFAAEIRDS | | | DDQVL | | | |
| | | SRQSVRMWLGT | | | QEML | | | |
| | | FDTAEEAARAY | | | | | | |
| | | DRAAYAMRGQI | | | | | | |
| | | AVLNFPAEA | | | | | | |
| Zm/G3516 | 6-70; 107- | KEGKYRGVRKR | 52 | 74% | KVELEC | 68 | 71% | |
| (32) | 122 | PWGKFAAEIRD | | | LDDRVL | | | |
| | | PERGGSRVWLG | | | EELL | | | |
| | | TFDTAEEAARA | | | | | | |
| | | YDRAAFAMKG | | | | | | |
| | | ATAVLNFPASG | | | | | | |
| Gm/G3519 | 13-77; 128- | CEVRYRGIRRRP | 53 | 72% | TFELEY | 69 | 66% | E |
| (34) | 143 | WGKFAAEIRDP | | | LDNKLL | | | |
| | | TRKGTRIWLGTF | | | EELL | | | |
| | | DTAEQAARAYD | | | | | | |
| | | AAAFHFRGHRA | | | | | | |
| | | ILNFPNEY | | | | | | |
| Gm/G3518 | 13-77; 135- | VEVRYRGIRRRP | 54 | 72% | TFELEY | 70 | 60% | E |
| (36) | 150 | WGKFAAEIRDP | | | FDNKLL | | | |
| | | TRKGTRIWLGTF | | | EELL | | | |
| | | DTAEQAARAYD | | | | | | |
| | | AAAFHFRGHRA | | | | | | |
| | | ILNFPNEY | | | | | | |
| Os/G3515 | 11-75; 116- | SSSSYRGVRKRP | 55 | 72% | KVELEC | 71 | 56% | |
| (38) | 131 | WGKFAAEIRDP | | | LDDKVL | | | |
| | | ERGGARVWLGT | | | EDLL | | | |
| | | FDTAEEAARAY | | | | | | |
| | | DRAAFAMKGAT | | | | | | |
| | | AMLNFPGDH | | | | | | |
| At/G1792 | 16-80; 117- | KQARFRGVRRR | 56 | 70% | VFEFEY | 72 | 87% | B, E, S |
| (40) | 132 | PWGKFAAEIRD | | | LDDKVL | | | |
| | | PSRNGARLWLG | | | EELL | | | |
| | | TFETAEEAARA | | | | | | |
| | | YDRAAFNLRGH | | | | | | |
| | | LAILNFPNEY | | | | | | |

TABLE 2

Conserved domains of Pti4 (TF family: AP2) and some closely
related sequences in the G28 clade

| | | | | | |
|---|---|---|---|---|---|
| Species/GID No., | | | | | Disease resistance |
| Accession No., or | AP2 Domain | | | % ID to | observed in |
| Identifier | Amino Acid | | SEQ ID NO: of | conserved AP2 | overexpressors |
| (SEQ ID NO:) | Coordinates | AP2 Domain | EDLL domain | domain of Pti4 | tested to date |

| | | | | | |
|---|---|---|---|---|---|
| Sl/Pti4 | 102-166 | KGRHYRGVRQRPWGKF | 97 | 100% | E, S |
| (4) | | AAEIRDPAKNGARVWL | | | |
| | | GTYETAEEAAIAYDKAA | | | |
| | | YRMRGSKAHLNFPHRI | | | |
| Gm/G3718 | 139-203 | KGKHYRGVRQRPWGKF | 98 | 87% | E, S |
| (74) | | AAEIRDPAKNGARVWL | | | |
| | | GTFETAEDAALAYDRA | | | |
| | | AYRMRGSRALLNFPLRI | | | |
| Gm/G3717 | 130-194 | KGKHYRGVRQRPWGKF | 99 | 86% | E, S |
| (76) | | AAEIRDPAKNGARVWL | | | |
| | | GTFETAEDAALAYDRA | | | |
| | | AYRMRGSRALLNFPLRV | | | |
| At/G28 | 144-208 | KGKHYRGVRQRPWGKF | 100 | 84% | B, E, S |
| (78) | | AAEIRDPAKNGARVWL | | | |
| | | GTFETAEDAALAYDRA | | | |
| | | AFRMRGSRALLNFPLRV | | | |
| Bo/G3659 | 130-194 | KGKHYRGVRQRPWGKF | 101 | 84% | E |
| (80) | | AAEIRDPAKNGARVWL | | | |
| | | GTFETAEDAALAYDRA | | | |
| | | AFRMRGSRALLNFPLRV | | | |
| Zm/G3856 | 140-204 | RGKHYRGVRQRPWGKF | 102 | 84% | E, S |
| (82) | | AAEIRDPAKNGARVWL | | | |
| | | GTYDSAEDAAVAYDRA | | | |
| | | AYRMRGSRALLNFPLRI | | | |
| Os/G3430 | 145-209 | RGKHYRGVRQRPWGKF | 103 | 84% | B, E, S |
| (84) | | AAEIRDPAKNGARVWL | | | |
| | | GTFDSAEEAAVAYDRA | | | |
| | | AYRMRGSRALLNFPLRI | | | |
| Os/G3848 | 149-213 | RGKHYRGVRQRPWGKF | 104 | 84% | B, E, S |
| (86) | | AAEIRDPAKNGARVWL | | | |
| | | GTFDTAEDAALAYDRA | | | |
| | | AYRMRGSRALLNFPLRI | | | |
| Zm/G3661 | 126-190 | RGKHYRGVRQRPWGKF | 105 | 84% | E |
| (88) | | AAEIRDPARNGARVWL | | | |
| | | GTYDTAEDAALAYDRA | | | |
| | | AYRMRGSRALLNFPLRI | | | |
| At/G1006 | 113-117 | KAKHYRGVRQRPWGKF | 106 | 83% | E, S |
| (90) | | AAEIRDPAKNGARVWL | | | |
| | | GTFETAEDAALAYDIAA | | | |
| | | FRMRGSRALLNFPLRV | | | |
| Bo/G3660 | 119-183 | KGKHYRGVRQRPWGKF | 107 | 81% | B, E, S |
| (92) | | AAEIRDPAKKGAREWL | | | |
| | | GTFETAEDAALAYDRA | | | |
| | | AFRMRGSRALLNFPLRV | | | |
| Ta/G3864 | 127-191 | RGKHFRGVRQRPWGKF | 108 | 81% | |
| (94) | | AAEIRDPAKNGARVWL | | | |
| | | GTFDSAEDAAVAYDRA | | | |
| | | AYRMRGSRALLNFPLRI | | | |
| At/G22 | 88-152 | KGMQYRGVRRRPWGKF | 109 | 80% | |
| (96) | | AAEIRDPKKNGARVWL | | | |
| | | GTYETPEDAAVAYDRA | | | |
| | | AFQLRGSKAKLNFPHLI | | | |

Disease resistance abbreviations: B - Botrytis; E - Erysiphe; S - Sclerotinia
Species abbreviations for Tables 1-2: At - Arabidopsis thaliana; Bo - Brassica oleracea; Gm - Glycine max; Os - Oryza sativa; Sl - Solanum lycopersicum; Ta - Triticum aestivum; Zm - Zea mays

### EXAMPLES

The data herein represent results obtained in experiments with polynucleotides and polypeptides that may be expressed in plants for the purpose of reducing yield losses that arise from biotic and abiotic stress. The invention, now being generally described, will be readily understood by reference to the following examples, which are included for purposes of illustration of certain aspects and embodiments of the present invention and are not intended to limit the invention. It will be recognized by one of skill in the art that a transcription factor that is associated with a particular first trait may also be associated with at least one other, unrelated and inherent second trait that was not predicted by the first trait.

### Example I

### Production of Plants Expressing TDR4 Under a Dexamethasone-Inducible System

Transformation. Transformation of Arabidopsis was performed by an Agrobacterium-mediated protocol based on the method of Bechtold and Pelletier (1998) Methods Mol. Biol. 82: 259-266. Unless otherwise specified, all experimental work was performed using the Columbia ecotype.

Plant preparation. Arabidopsis seeds were sown on mesh covered pots. The seedlings were thinned so that 6-10 evenly spaced plants remained on each pot 10 days after planting. The primary bolts were cut off a week before transformation to break apical dominance and encourage auxiliary shoots to form. Transformation was typically performed at 4-5 weeks after sowing.

Bacterial culture preparation. Agrobacterium stocks were inoculated from single colony plates or from glycerol stocks and grown with the appropriate antibiotics and grown until saturation. On the morning of transformation, the saturated cultures were centrifuged and bacterial pellets are re-suspended in Infiltration Media (0.5×MS, 1×B5 Vitamins, 5% sucrose, 1 mg/ml benzylaminopurine riboside, 200 μl/L Silwet L77) until an A600 reading of 0.8 was reached.

Transformation and seed harvest. The Agrobacterium solution was poured into dipping containers. All flower buds and rosette leaves of the plants were immersed in this solution for 30 seconds. The plants were laid on their side and wrapped to keep the humidity high. The plants were kept this way overnight at 4° C. and then the pots were turned upright, unwrapped, and moved to the growth racks.

The plants were maintained on the growth rack under 24-hour light until seeds were ready to be harvested. Seeds were harvested when 80% of the siliques of the transformed plants were ripe (approximately 5 weeks after the initial transformation). This seed was deemed T0 seed, since it was obtained from the T0 generation, and was later plated on selection plates (either kanamycin or sulfonamide). Resistant plants that were identified on such selection plates comprise the T1 generation.

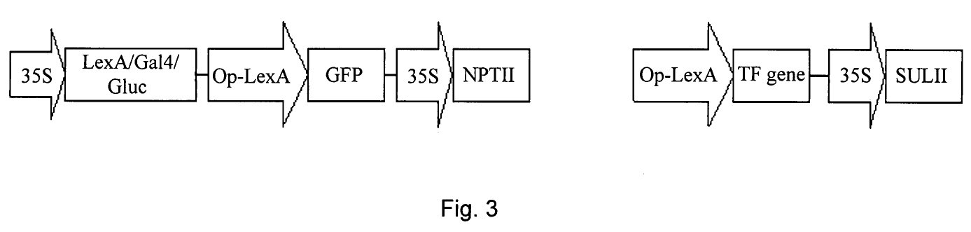

Establishment of the dexamethasone-inducible TDR4 Arabidopsis line. A kanamycin-resistant line expressing the activator construct described in FIG. 3 was produced by the methods specified above. Homozygous T3 progeny were selected from this line and verified to produce the desired dexamethasone-inducible expression pattern of the GFP reporter gene. A homozygous line was then transformed with the target construct carrying TDR4 under the control of the LexA operator. Plant lines were made homozygous for the TDR4 transgene and single insertion lines were identified by Southern blotting using standard molecular methods (see Current Protocols in Molecular Biology, Ausubel et al. eds., Current Protocols, a joint venture between Greene Publishing Associates, Inc. and John Wiley & Sons, Inc., (supplemented through 2000); “Ausubel”).

### Example II

### Mutagenesis

Dexamethasone-inducible TDR4 Arabidopsis seeds were mutagenized with ethyl methane sulfonate (EMS) as described by Redei and Koncz (1992) “Classical Mutagenesis”, In C Koncz, N-H Chua, J. Schell, eds, Methods in Arabidopsis Research. World Scientific, Singapore, pp 16-82. Seeds were imbibed in H₂O overnight at room temperature, than shaken in 50 ml Falcon tubes with 25 ml of 50 mM EMS for 8 hours at room temperature and washed 10 times with sterile distilled water after EMS treatment. For a final wash step, seeds were shaken in sterile distilled water overnight at room temperature. The next morning, 0.1% agarose was added to the Falcon tube and seeds were stored at 4° C. for 48 h.

Seeds were then planted into 2×5 cell flats filled with Sunshine Soil Mix (+entomite). About 100 seeds were placed into each cell. After germination, the number of albino plants was scored to estimate the mutation level. Plants grew at 20° C. and 24 h light, were fertilized weekly and pools of 100 plants=1 cell were bagged and harvested. One hundred 10-cell flats were grown and thus 1000 pools were generated.

### Example III

### Suppressor Mutation Screen

Plant lines constitutively expressing TDR4 are severely stunted, while the dexamethasone-inducible TDR4 lines when not exposed to dexamethasone have a growth phenotype significantly more similar to wild-type plants than 35S::TDR4 overexpressing lines in that the dexamethasone-inducible TDR4 lines had fewer or reduced adverse morphological or developmental effects than the wild-type controls. Use of the dexamethasone inducible system therefore allowed for generation of T2 seeds for mutagenesis, which was not possible with the 35S::TDR4 lines due to severe growth defects and infertility. Screening of the M2 pools was therefore conducted on plates containing 5 μM dexamethasone in order to reveal the growth retardation phenotype. Other components of the medium were 50% MS salts (Murashige and Skoog (1962) Physiol. Plant. 15: 473-497), 1% sucrose, and 0.05% MES (2-(N-Morpholino)ethanesulfonic acid hydrate). About 1200 seeds per pool were screened. Seeds were surface sterilized in the following manner: (1) 5 minute incubation with mixing in 70% ethanol; (2) 20 minute incubation with mixing in 30% bleach, 0.01% Triton X-100; (3) five rinses with sterile water. The seeds were resuspended in 0.1% sterile agarose and stratified at 4° C. for 2-4 days. Two hundred ethanol/bleach sterilized seeds were plated onto one 150×15 Petri dish which amounts to 6 plates/pool. Plates were transferred to 22° C. germination chambers with 24 h light. One plate with the non-mutagenized TDR4 line as well as one plate with a line containing a target construct lacking the TDR4 transgene were also plated as controls. Under these conditions, the dexamethasone-inducible TDR4 lines showed obvious growth retardation in comparison to the control plants lacking the TDR transgene. After 12-13 days, plates were examined for seedlings with relatively normal morphology. These plants were then screened for retention of GFP fluorescence, to eliminate mutations in the activator construct. Putative mutants were transferred to soil to collect seed.

### Example IV

### Sequencing of the TDR4 Transgene

While the selected M2 plants were growing in soil, leaf samples were taken for DNA extraction and PCR analysis, performed by standard methods (see, for example, Ausubel, supra). The TDR4 transgene sequence was amplified using a forward primer within the TDR 5′ untranslated region (SEQ ID NO: 7) and a 3′ primer within the cloning vector (SEQ ID NO: 8). The resulting PCR product was sequenced to identify any mutations within the transgene.

Sequences were analyzed using Sequencher DNA sequence analysis software (Gene Codes Corporation, Ann Arbor, Mich.). Plants that harbored no mutations in the TDR4 transgene coding sequence were presumed to carry second site mutations, or mutations in the LexA operator fused to the TDR4 gene, and were not analyzed further. Some putative mutants showed double peaks at possible mutation sites, indicating heterozygosity. The M3 generation was grown for these EMS lines and DNA samples were taken from 5-8 plants to identify the line with the mutation either by a restriction digestion with CAPS markers, when possible or by sequencing of TDR4. Lines harboring the mutation where further analyzed as described below.

### Example V

### Disease Assays

M3 progeny of the M2 mutant plants isolated above were tested in disease assays in the presence of 5 μM dexamethasone to determine whether the mutated TDR4 would still provide disease resistance.

Resistance to Sclerotinia sclerotiorum and Botrytis cinerea were assessed in plate-based assays. Unless otherwise stated, all experiments were performed with the Arabidopsis thaliana ecotype Columbia (Col-0). Control plants for assays on lines containing direct promoter-fusion constructs were wild-type plants or Col-0 plants transformed with an empty transformation vector.

Prior to plating, seed for all experiments were surface sterilized in the following manner: (1) 5 minute incubation with mixing in 70% ethanol; (2) 20 minute incubation with mixing in 30% bleach, 0.01% Triton X-100; (3) five rinses with sterile water. Seeds were resuspended in 0.1% sterile agarose and stratified at 4° C. for 2-4 days.

Sterile seeds were sown on starter plates (15 mm deep) containing the following medium: 50% MS solution, 1% sucrose, 0.05% MES, and 1% Bacto-Agar. 40 to 50 seeds were sown on each plate. Plates were incubated at 22° C. under 24-hour light (95-110 μE m-2 s-1) in a germination growth chamber. On day 10, seedlings were transferred to assay plates (25 mm deep plates with medium minus sucrose, plus 5 μM dexamethasone). On day 14, seedlings were inoculated (specific method below). After inoculation, plates were put in a growth chamber under a 12-hour light/12-hour dark schedule. Light intensity was lowered to 70-80 μE m-2 s-1 for the disease assay.

Sclerotinia inoculum preparation. A Sclerotinia liquid culture was started three days prior to plant inoculation by cutting a small agar plug (¼ sq. inch) from a 14- to 21-day old Sclerotinia plate (on Potato Dextrose Agar; PDA) and placing it into 100 ml of half-strength Potato Dextrose Broth. The culture was allowed to grown in the Potato Dextrose Broth at room temperature under 24-hour light for three days. On the day of seedling inoculation, the hyphal ball was retrieved from the medium, weighed, and ground in a blender with water (50 ml/gm tissue). After grinding, the mycelial suspension was filtered through two layers of cheesecloth and the resulting suspension was diluted 1:5 in water. Plants were inoculated by spraying to run-off with the mycelial suspension using a Preval aerosol sprayer (Precision-Valve Corporation, Yonkers, N.Y.).

Botrytis inoculum preparation. Botrytis inoculum was prepared on the day of inoculation. Spores from a 14- to 21-day old plate were resuspended in a solution of 0.05% glucose, 0.03M KH₂PO₄ to a final concentration of 10⁴ spores/ml. Seedlings were inoculated with a Preval aerosol sprayer, as with Sclerotinia inoculation.

Data Interpretation. After the plates were evaluated, each line was given one of the following overall scores:

(++) Substantially enhanced resistance compared to controls. The phenotype was very consistent across all plates for a given line.

(+) Enhanced resistance compared to controls. The response was consistent but was only moderately above the normal levels of variability observed for that assay.

(wt) No detectable difference from wild-type controls.

(−) Increased susceptibility compared to controls. The response was consistent but was only moderately above the normal levels of variability observed for that assay.

(−−) Substantially impaired performance compared to controls. The phenotype was consistent and growth was significantly above the normal levels of variability observed for that assay.

(n/d) Experiment failed, data not obtained, or assay not performed.

### Example VI

### Confirmation of Mutations

It is possible that a line containing a TDR4 transgene mutation also harbors another mutation that affects disease resistance in general or TDR4 function specifically. Therefore, the mutant alleles identified in the morphology and disease screens will be amplified from the transgenic plants, re-cloned behind the 35S constitutive promoter, and transformed into wild-type Col-0 Arabidopsis plants. T1 transformants will be selected on kanamycin and T2 plants will be tested for disease resistance. Disease resistance seen in multiple, independently-transformed lines with normal morphology will demonstrate a direct correlation between the mutant TDR4 allele and disease resistance without severe growth penalty.

### Example VII

### Mutagenesis of Pti4

Pti4, SEQ ID NO: 4, is an AP2 domain transcription factor that produces disease resistance when expressed under a constitutive Cauliflower Mosaic Virus 35S promoter. However, plants expressing Pti4 under a constitutive promoter are stunted, dark green, and late flowering. Because 35S::Pti4 transgenic plants are fertile, a variant of the method described above for TDR4 is used. Homozygous 35S::Pti4 transgenic plants are produced by standard transformation techniques as described in Example I above. Either a 35S::Pti4 direct promoter fusion construct or a two-component approach could be used. The resulting plants are mutagenized with EMS as described in Example II above. M2 plants are then planted either on sterile medium in the absence of dexamethasone, or on soil, and are screened visually for plants with reduced stunting, dark green color, or flowering delay. Such plants are saved for seed, and leaf tissue is harvested for amplification and sequencing of the Pti4 transgene using standard methods. Plants harboring mutations in the Pti4 transgene are saved for seed, and their progeny are assayed for disease resistance as described in Example V above. For plants showing disease resistance, the altered Pti4 transgene is cloned and re-transformed into plants to confirm the beneficial phenotype.

### Example VIII

### The Present Methods May be Used to Select for Beneficial Sequence Changes to Other Transcription Factor Families

The methods described above represent an improvement on a basic suppressor mutagenesis screen with plant lines ectopically expressing transcription factors under the regulatory control of the 35S promoter. The present methods provide an approach to overexpress deleterious or near-lethal transcription factors that, when transformed into plants using a constitutive regulation means, produce stunted or developmentally retarded plants with reduced or no fertility. A strong selection for beneficial sequence changes (i.e., mutations that do not produce severely and adversely affected plants) may be applied since all of the plants lacking those mutations grow extremely slowly or die when dexamethasone is applied.

The same approach may be taken with transcription factor polynucleotides and their predicted polypeptides that may produce moderate highly deleterious effects in plants when the sequences are overexpressed in plants. A listing of Arabidopsis sequences for which any deleterious or undesirable developmental or morphological effects of constitutive overexpression may be mitigated to some degree are provided as SEQ ID NOs: 110-973. It is expected that the same approach may be employed with sequences that are orthologous to SEQ ID NOs: 110-973 and which function in the same regard as the Arabidopsis sequences.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the Claims. Modifications that become apparent from the foregoing description and accompanying figures fall within the scope of the following Claims.

What is claimed is:

1. A method for producing a plant that has greater biotic resistance or abiotic stress tolerance than a first control plant, and fewer or reduced adverse morphological or developmental effects than a second control plant, the method steps comprising:
(a) providing a two component expression system comprising:
(i) a target nucleic acid construct that encodes a transcription factor polypeptide; and
(ii) an activator nucleic acid construct encoding a steroid-binding domain of a glucocorticoid receptor;
(b) introducing the two component expression system into a target plant;
(c) selecting transgenic plant lines homozygous for the target nucleic acid construct and the activator nucleic acid construct;
(c) mutagenizing the transgenic plant lines to produce a pool of mutagenized transgenic plant lines comprising sequence variants of the transcription factor polypeptide; and
(d) selecting one or more of the mutagenized transgenic plant lines that have:
(i) greater biotic resistance or abiotic stress tolerance than the first control plant, wherein the first control plant does not overexpress the transcription factor polypeptide; and
(ii) fewer or reduced adverse morphological or developmental effects as compared to the second control plant, wherein the second control plant constitutively overexpresses the transcription factor polypeptide.

2. The method of claim 1, wherein the target nucleic acid construct comprises a LexA operator that regulates expression of the transcription factor polypeptide; and
the activator nucleic acid construct comprises a LexA DNA binding domain fused to a GAL4 activation domain and the steroid-binding domain of the glucocorticoid receptor.

3. The method of claim 1, wherein the biotic stress resistance is tolerance to a fungal plant disease.

4. The method of claim 3, wherein the fungal plant disease is caused by a biotrophic or necrotrophic pathogen.

5. The method of claim 3, wherein the fungal plant disease is caused by Botrytis, Erysiphe, or Sclerotinia.

6. The method of claim 1, wherein the transcription factor polypeptide is a homolog of SEQ ID NO: 4 that comprises a conserved AP2 domain having at least 80% amino acid sequence identity to a conserved AP2 domain of amino acids 102-166 of SEQ ID NO: 4.

7. The method of claim 6, wherein the conserved AP2 domain has at least 84% amino acid sequence identity to a conserved AP2 domain of amino acids 16-80 of SEQ ID NO: 2.

8. The method of claim 1, wherein the transcription factor polypeptide comprises SEQ ID NO: 4.

9. The method of claim 1, wherein the transcription factor polypeptide is a homolog of SEQ ID NO: 2 that comprises a conserved AP2 domain that has at least 76% amino acid sequence identity to a conserved AP2 domain of amino acids 16-80 of SEQ ID NO: 2.

10. The method of claim 9, wherein the conserved AP2 domain has at least 84% amino acid sequence identity to a conserved AP2 domain of amino acids 16-80 of SEQ ID NO: 2.

11. The method of claim 9, wherein the conserved AP2 domain has at least 93% amino acid sequence identity to a conserved AP2 domain of amino acids 16-80 of SEQ ID NO: 2.

12. The method of claim 1, wherein the one or more of the mutagenized transgenic plant lines produces greater yield than the first control plant.

13. The method of claim 1, wherein the target plant is generated by introducing the activator nucleic acid construct into a first plant, a second plant is selected that is homozygous for the activator nucleic acid construct, the second plant is transformed with the target nucleic acid construct to generate a third plant, and a fourth plant is selected that is homozygous for both the activator and target nucleic acid constructs.

14. The method of claim 1, wherein the transcription factor polypeptide comprises any of SEQ ID NOs: 2, 4, 12, 14, 16, 18, 20, 22, 24, 26, 28, 30, 32, 34, 36, 38 or 40, or 41-72, or 74, 76, 78, 80, 82, 84, 86, 88, 90, 92, 94, or 96, or any of SEQ ID NO: 2n−1, where n=56-487.

15. A transgenic plant produced according to the method of claim 1;
wherein the transgenic plant comprises and is homozygous for the target nucleic acid construct and the activator nucleic acid construct; and
wherein the transgenic plant has greater biotic resistance or abiotic stress tolerance than the first control plant of claim 1, and fewer or reduced adverse morphological or developmental effects than a second control plant of claim 1.

16. A transgenic seed produced by the transgenic plant of claim 15, wherein the transgenic seed comprises and is homozygous for the target nucleic acid construct and the activator nucleic acid construct.
